(19) United States
(12) Patent Application Publication
(10) Pub. No.: US 2023/0101007 A1
(43) Pub. Date: Mar. 30, 2023

(54) Heel Warming Sock

(71) Applicants: Levyn Polanco, Sylmar, CA (US); Jezebel Polanco, Sylmar, CA (US)

(72) Inventors: Levyn Polanco, Sylmar, CA (US); Jezebel Polanco, Sylmar, CA (US)

(21) Appl. No.: 17/934,773

(22) Filed: Sep. 23, 2022

Related U.S. Application Data
(60) Provisional application No. 63/249,315, filed on Sep. 28, 2021.

(51) Int. Cl. A41D 13/005; A41B 11/02

(52) U.S. Cl. CPC A41D 13/0051; A41B 11/02

(57) ABSTRACT

A foot warming device for warming a foot of an infant for medical procedures, such as a heel stick procedure. A warming component is integrated into or is insertable into a sock or bootie. The warming component is located within the sock to warm a heel, a sole, a dorsum, or a combination of these parts of an infant's foot. Heat is generated exothermically from the warming component when a thermochemical composition is exposed to air or is agitated mechanically. The generated heat warm the adjacent part of the foot for long enough to improve blood flow to the area. The generated heat dissipates naturally as the thermochemical composition is consumed.

### CROSS-REFERENCE TO RELATED APPLICATION

The present application claims priority to, and the benefit of, U.S. Provisional Application No. 63/249,315, which was filed on Sep. 28, 2021 and is incorporated herein by reference in its entirety.

### FIELD OF THE INVENTION

The present invention generally relates to an infant foot warmer, and more specifically to a sock or foot covering configured to warm the heel of an infant to assist in heel stick blood draws. Accordingly, the present specification makes specific reference thereto. However, it is to be appreciated that aspects of the present invention are also equally amenable to other like applications, devices, and methods of manufacture.

### BACKGROUND

Blood draws for medical testing are never easy, especially when performed on an infant or newborn. There are many issues that can arise with this procedure. For example, infant heels must be properly warmed to improve blood flow to the region before obtaining a sample. If this is not done, the sample quality may not be good enough for testing. Redraws are time consuming and result in excessive use of expensive supplies. If circulation is restricted and a redraw is required, infant heels can become bruised or injured from the multiple needle sticks. This not only causes further pain and agitation to the patient, but anxiety for the child's parents. These issues make it difficult to provide quality care and lead to decreases in patient satisfaction. The medical field needs an improved infant foot warming device which will circumvent these obstacles and provide peace of mind for both patients and medical staff.

A newborn heel stick test is a routine medical procedure performed on babies 24 to 48 hours after birth. The test screens for serious health conditions, including many metabolic and hormone disorders that don't show symptoms at birth but can be dangerous if untreated. A sharp device known as a lancet is used to prick the baby's heel to obtain blood sample to obtain a tiny sample of blood for analysis. A heel stick is the most common and minimally invasive method to draw capillary blood from an infant for medical testing. Capillaries are tiny blood vessels which carry blood to the tissues and connect arteries to the veins.

Heel sticks are relatively safe if the puncture site is limited to the medial and lateral planter aspects of each heel pad, specifically medial to a visual line drawn from the middle of the big toe extending posterior to the heel or lateral to a line drawn from between the fourth and fifth toes and extending posterior to the heel. Repeated punctures, bruising, or erythema can limit the available area on the heel for punctures. This is exacerbated in premature infants who may have blood drawn multiple times or very tiny heels. Though heel warming is commonly performed to increase blood flow in the capillaries. Using warm water submersion is risky unless the temperature is tightly controlled. Chemical heat packs and patches offer a temperature-controlled, safer, but more expensive alternative.

Existing infant heel warmers work generally by increase blood flow to infant's heel area, minimizing potential bruising, infection or cartilage damage caused by frequent heel sticks from blood tests. An infant heel warmer is a type of disposable device made up of a non-toxic substance. The most common type make use of gel components for increasing the body's temperature. High-quality infant heel warmers reach the right temperature within just a minute without any complicated procedure to activate them. It only takes squeezing the pouch containing the gel to activate them. These warmers employ lift-and-stick tape for easy application and removal. When placed in the sole and heel area, an exothermic reaction occurs which immediately raises the body temperature. The rise in temperature only lasts for a few minutes but is typically enough to boost the infant's blood circulation, which helps providers to collect a blood sample of the infant.

Accordingly, there is a great need for a flexible infant foot warmer that can be continuously worn even after heat is no longer generated. There is also a need for a way for warming the soles and heels of babies feet to provide increased blood flow. Similarly, there is a need for a product to assist medical professionals in drawing blood on infants for medical tests. There is also a need for a warming device that limits excessive poking and injury of infant heels to obtain quality blood samples. Further, there is a need for a better war to warm an infant's foot quickly and safely for medical procedures.

In this manner, the improved heel and foot warming sock device of the present invention accomplishes all of the forgoing objectives, thereby providing an easy solution for warming the soles and heels of infants. A primary feature of the present invention is an exothermic composition that can be integrated into a sock or bootie. The present invention is designed to assist medical professionals in drawing blood on infants. Finally, the improved foot warming sock device of the present invention is capable of warming an infant's foot quickly and safely to help limit complications associated with heel stick testing on infants.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The subject matter disclosed and claimed herein, in one embodiment thereof, comprises a foot warming device. The foot warming device is configured to warm at least a heel or sole area of an infant's foot via an exothermic chemical reaction. The foot warming device is designed to quickly and safely warm parts on an infant's foot used for heel stick blood draws and for foot warming.

The foot warming device comprises a sock component and a warming component. The sock component may be a sock or bootie sized to fit a newborn or an infant. The sock component comprises a heel portion, a sole portion, and a dorsal portion. The warming component is positional within the sock component. The sock component may further comprise a plurality of pockets on the inside of the sock component adjacent to the heel portion, the sole portion, the dorsal portion, or any combination thereof.

The warming component comprises an air permeable container and a thermochemical composition. The thermochemical composition is retained within the air permeable container. The air permeable container is a porous pouch configured to retain the thermochemical composition while simultaneously allowing air to permeate the pouch. The warming component may be integrated into or adhered to an inside surface of the inside of the sock component. The warming component may be located adjacent to the heel portion, the sole portion, or the dorsal portion on the inside of the sock component.

The thermochemical composition is formulated to generate heat exothermically when exposed to air. The thermochemical composition is a formulation of iron powder, water, a heat distributing material, a catalyst, and an insulator. The heat distributing material is typically activated carbon or activated charcoal. The catalyst is typically sodium chloride (NaCl). The insulator is typically vermiculite or polyacrylate. The thermochemical composition formulation may further comprise a filler, such as cellulose.

The warming component further comprises an airtight base layer and an airtight cover. The airtight base layer is positional between the air permeable container and an inner surface of the sock component. The airtight cover is configured to encapsulate the air permeable container between the airtight cover and the airtight base layer. The airtight cover is removable or breachable to expose the air permeable container to air to initiate the exothermic chemical reaction.

The foot warming device may further comprise a plurality of warming components. Each warming component comprises an air permeable container, a thermochemical composition, an airtight base layer and an airtight cover. The plurality of warming components may be located adjacent to the heel portion, the sole portion, the dorsal portion on the inside of the sock component, or any combination thereof.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a cross-sectional side view of one potential embodiment of a foot warming device of the present invention for warming a heel or foot of an infant in accordance with the disclosed architecture.

FIG. 2 illustrates a perspective view of one potential component of a warming component of the foot warming device of the present invention in accordance with the disclosed architecture.

FIG. 3 illustrates a cross-sectional side view of one potential embodiment of the warming component of the foot warming device of the present invention in accordance with the disclosed architecture.

FIG. 4 illustrates a perspective view of one potential embodiment of the foot warming device of the present invention in accordance with the disclosed architecture.

FIG. 5 illustrates a cut-away view of one potential embodiment of the foot warming device of the present invention in accordance with the disclosed architecture.

FIG. 6 illustrates a cut-away view of one potential embodiment of the foot warming device of the present invention in accordance with the disclosed architecture.

FIG. 7 illustrates a cross-sectional side view of one potential view of the warming component of the foot warming device of the present invention in accordance with the disclosed architecture.

FIG. 8 illustrates a cross-sectional side view of one potential embodiment of the warming component of the foot warming device of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They do not intend as an exhaustive description of the invention or do not limit the scope of the invention. Additionally, an illustrated embodiment need not have all the aspects or advantages shown. Thus, in other embodiments, any of the features described herein from different embodiments may be combined.

The present invention, in one exemplary embodiment is a foot warming device. The foot warming device may have a general appearance of a sock or fitted shoe. The material can be similar to that of traditional squared heel warmers, but the form-fitting design of the present invention will hold heat better. The sock or bootie, which retain the same texture and elasticity as regular infant sock, wrap around a newborn's foot. They have a built-in thermal technology but are not bulky.

Thin thermal packs pass warmth along the soles and heels of the infant. These heating pads may be divided between the top and bottom of the sock by an open strip so that the toes are free from the thermal element. The heating properties are activated by massaging or squeezing the device or exposing it to air. The foot warming sock can be used by medical professionals on newborn patients to improve circulation in the heel region for blood draws. This product will make obtaining quality blood samples for medical testing much easier with less poking and injury to the infant. The foot warming sock will provide the right angles for optimum test results and quality patient care.

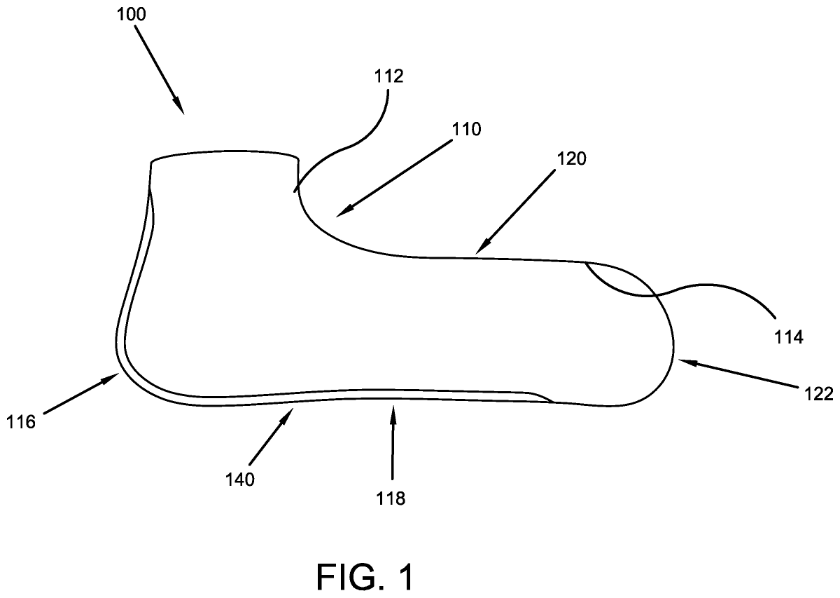

Referring initially to the drawings, FIGS. 1-8 illustrate a foot warming device 100. The foot warming device 100 is configured to heat at least a heel or sole area of an infant's foot via an exothermic chemical reaction. The foot warming device 100 is designed to quickly and safely warm parts of an infant's foot used for heel stick blood draws and for foot warming.

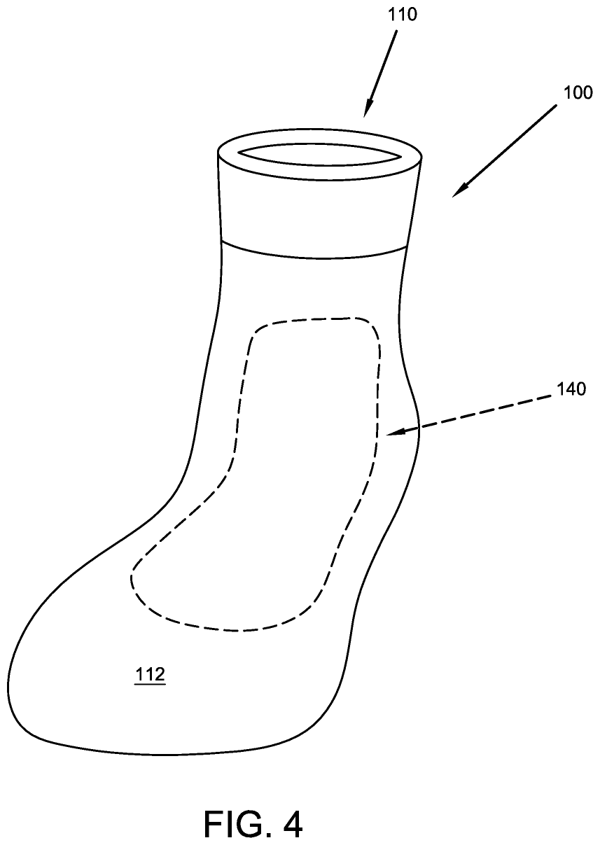

As illustrated in FIGS. 1 and 4, the foot warming device 100 comprises a sock component 110 and a warming component 140. The sock component 110 may be a sock or bootie adapted and sized to fit a newborn or an infant foot. The sock component 110 comprises an inner surface 112 and an outer surface 114. The sock component 110 may be manufactured from a wide variety of materials, such as cotton, wool, nylon, acrylic, polyester, olefins, and the like. The sock component 110 further comprises a heel portion 118, a sole portion 118, a dorsal portion 120, and a toe portion 122.

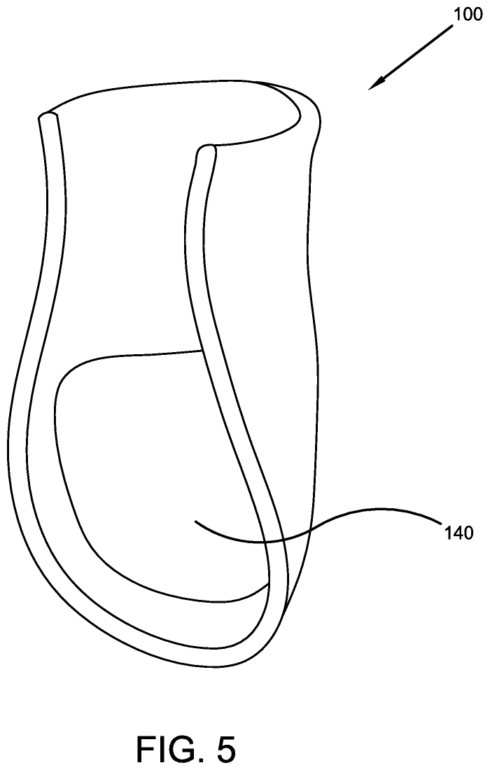

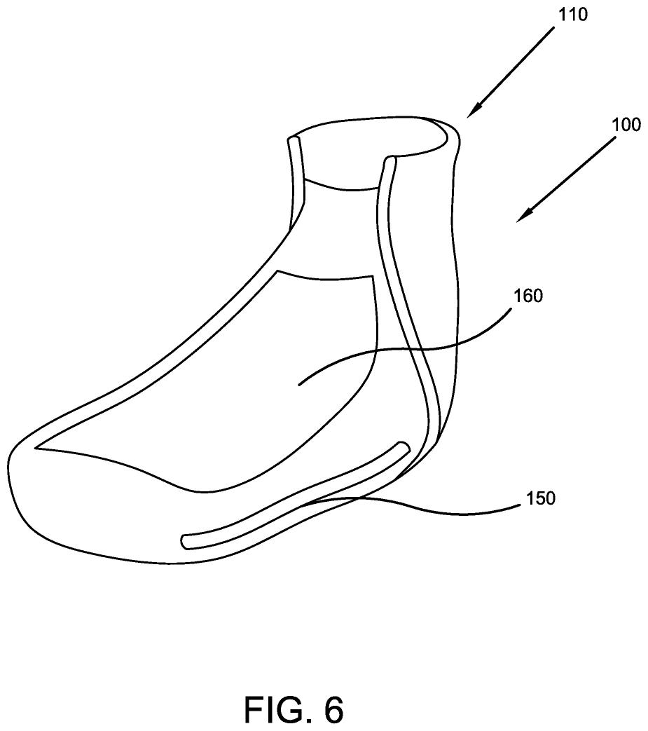

The warming component 140 is adapted for positioning or locating within an interior of the sock component 110. As illustrated in FIGS. 5 and 6, the warming component 140 may be interiorly positioned adjacent to the heel portion 118, the sole portion 118, or the dorsal portion 120 within the sock component 110. The warming component 140 may be integrated directly into the inner surface 112 of the sock component 110 or may be adhered to the inner surface 112 of the sock component 110 mechanically or with an adhesive.

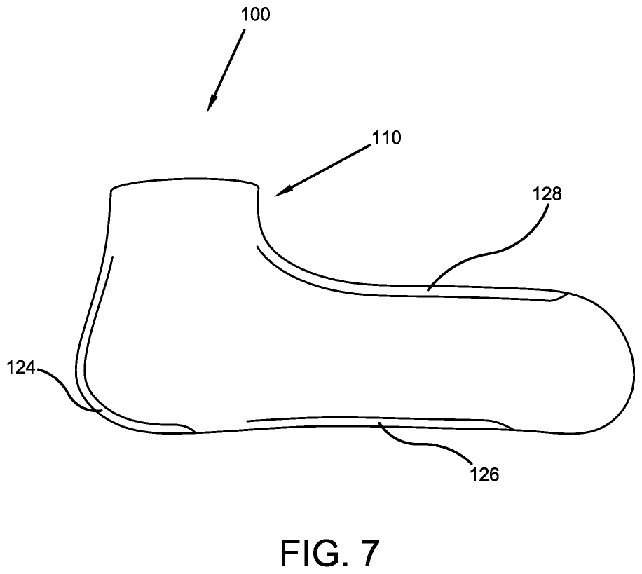

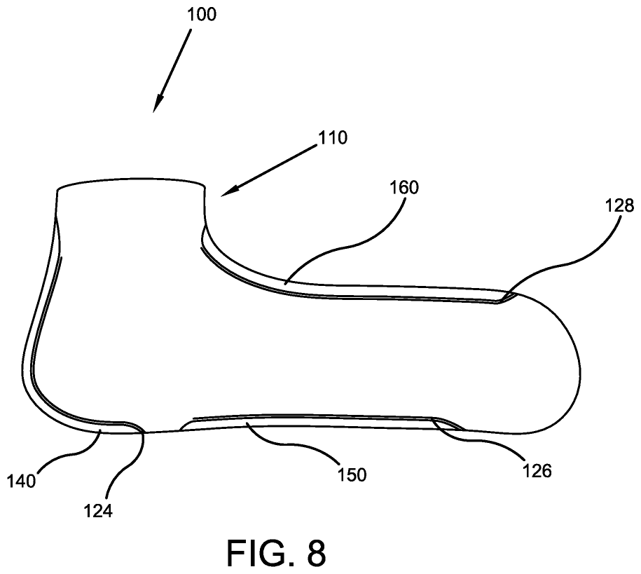

Alternatively, as illustrated in FIGS. 7 and 8, the sock component 110 may further comprise a plurality of pockets 124, 126, and 128. The plurality of pockets 124, 126, and 128 are located on the inside of the sock component 110. A heel pocket 126 is adjacent to the heel portion 116, a sole pocket 124 is adjacent to the sole portion 118, and a dorsal pocket 128 is adjacent to the dorsal portion 120 of the sock component 110 on the inner surface 112. The plurality of pockets 124, 126, and 128 are configured to hold the warming component 140 in one embodiment of the invention. The warming component 140 would then be removable and replaceable in any of the plurality of pockets 124, 126, and 128.

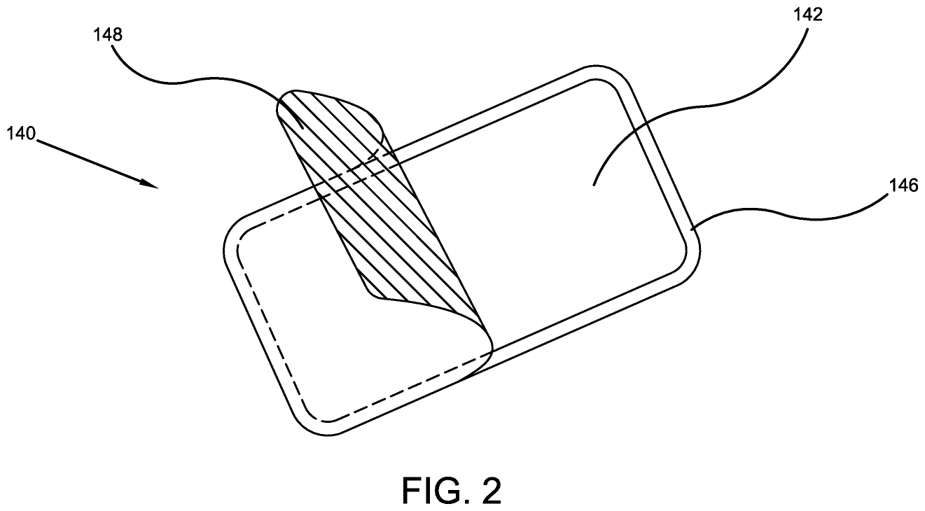

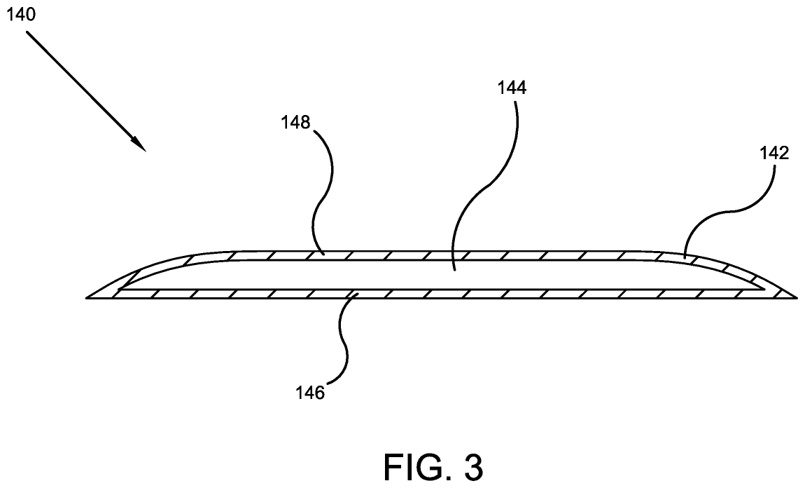

As illustrated in FIGS. 2 and 3, the warming component 140 comprises an air permeable container 142 and a thermochemical composition 144. The thermochemical composition 144 is retained within the air permeable container 142. The air permeable container 142 is a porous pouch configured to retain the thermochemical composition 144 while allowing air to permeate the air permeable container 142. The air permeable container 142 may be a pouch manufactured from polypropylene, a breathable plastic, or a breathable non-woven material. Polypropylene is advantageous as this material allows air to permeate the thermochemical composition 144 while holding in moisture. The air permeable container 142 is typically attached to or integrated into the inside surface 114 of the sock component 110.

The thermochemical composition 144 is formulated to generate heat exothermically when exposed to air. The thermochemical composition 144 is typically a formulation of iron powder, water, a heat distributing material, a catalyst, and an insulator. The thermochemical composition 144 generates heat via an exothermic reaction that, in essence, creates iron oxide or rust in a chemical process called oxidation.

The iron powder or particles may be varied in amount and surface area to control the speed and length of the reaction. The greater the surface area of the iron, the more the iron can react with oxygen to produce heat. The air permeable container 142 material also affects the performance of the foot warming device 100. The iron powder and other ingredients are contained in a material that has specific permeability characteristics. If the air permeable container 142 admits more oxygen, the reaction occurs more quickly. In one formulation, once activated, the thermochemical composition 144 is formulated to generate heat of between 100-110 degrees Fahrenheit for up to 20 minutes. However, the temperature generated may be up to 135 degrees Fahrenheit for an hour or more.

The heat distributing material is typically activated carbon or activated charcoal. Activated charcoal as a porous material that holds the water necessary for the oxidizing reaction to occur and helps to evenly disperse the heat produced. The catalyst is typically a salt, such as sodium chloride (NaCl) which in combination with the water helps speed the reaction. The insulator is typically vermiculite or polyacrylate which expands when heated and retains the heat. The insulator also helps diffuse the iron powder so the filings don't burn too quickly. The thermochemical composition formulation may further comprise a filler, such as cellulose.

The warming component 140 further comprises an airtight base layer 146 and an airtight cover 148. The airtight base layer 146 and the airtight cover 148 are typically manufactured from polymers, such as plastic polyethylene or similar nonpermeable plastic. The airtight base layer 146 is positional between the air permeable container and an inner surface 114 of the sock component 110. The airtight base layer 146 may be fixed in place mechanically or with an adhesive. The airtight cover 148 is configured to encapsulate or sandwich the air permeable container 142 between the airtight cover 148 and the airtight base layer 146.

The airtight cover 148 is removable to expose the air permeable container 142 to air by peeling it off or otherwise rupturing it. When the airtight cover 148 is removed, oxygen drifts across the air permeable container 142 covering to react with the thermochemical composition 144. With salt and water present, the oxygen reacts with the iron powder located inside to form iron oxide (Fe₂O₃) and release heat. Eventually, all the iron oxidizes and the heat dissipates.

As illustrated in FIG. 8, the foot warming device 100 may further comprise a plurality of warming components 140, 150, and 160. Each warming component 140, 150, and 160 comprises an air permeable container 142, a thermochemical composition 144, an airtight base layer 146 and an airtight cover 148. The plurality of warming components 140, 150, and 160 may be located adjacent to the heel portion 116, the sole portion 118, the dorsal portion 120 on the inside of the sock component, or any combination thereof. The plurality of warming components 140, 150, and 160 may be attached to the inner surface 114 of the sock component 110 or may be housed in the respective pockets 124, 126, and 128.

Alternatively, the thermochemical composition 144 may comprise a supersaturated solution of sodium acetate. A metal disc may be used to release a small number of crystals of sodium acetate which act as nucleation sites for the crystallization of the sodium acetate into a hydrated salt. Energy is released exothermically from the crystal lattice. The thermochemical composition 144 can be placed in boiling water and the sodium acetate can be dissolved again for reuse.

Notwithstanding the forgoing, the foot warming device 100 of the present invention can be any suitable size, shape, and configuration as is known in the art without affecting the overall concept of the invention, provided that it accomplishes the above stated objectives. One of ordinary skill in the art will appreciate that the shape and size of the foot warming device 100 and its various components, as show in the FIGS. are for illustrative purposes only, and that many other shapes and sizes of the foot warming device 100 are well within the scope of the present disclosure. Although dimensions of the foot warming device 100 and its components (i.e., length, width, and height) are important design parameters for good performance, the foot warming device 100 and its various components may be any shape or size that ensures optimal performance during use and/or that suits user need and/or preference. As such, the foot warming device 100 may be comprised of sizing/shaping that is appropriate and specific in regard to whatever the foot warming device 100 is designed to be applied.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A foot warming device comprising:
a sock component;
a warming component adapted for positioning within the sock component, the warming component comprising an air permeable container and a thermochemical composition retained within the air permeable container; and
wherein the thermochemical composition generates heat upon exposure to air.

2. The foot warming device of claim 1, wherein the sock component is a booty adapted for a newborn foot.

3. The foot warming device of claim 1, wherein the warming component is positioned adjacent to a heel portion of the sock component.

4. The foot warming device of claim 1, wherein the air permeable container is a polypropylene pouch.

5. The foot warming device of claim 1, wherein the air permeable container is a non-woven material pouch.

6. The foot warming device of claim 1, wherein the warming component is integrated into an interior of the sock component.

7. The foot warming device of claim 1, wherein the warming component is adhered to an interior of the sock component with an adhesive.

8. The foot warming device of claim 1, wherein the thermochemical composition is formulated to generate heat exothermically.

9. The foot warming device of claim 1, wherein the thermochemical composition is comprises iron powder, water, a heat distributing material, a catalyst, and an insulator.

10. The foot warming device of claim 1, wherein once activated, the thermochemical composition generates heat for up to twenty minutes.

11. The foot warming device of claim 1, wherein once activated, the thermochemical composition generates heat at a temperature ranging from between 100 and 110 degrees Fahrenheit.

12. A foot warming device to improve blood flow to a heel comprising:
a sock component;
a warming component adapted for positioning within the sock component, the warming component comprising an air permeable container, a thermochemical composition retained within the air permeable container, an airtight base layer positional between the air permeable container and an inner surface of the sock component, and an airtight cover configured to encapsulate the air permeable container between the airtight cover and the airtight base layer; and
wherein the thermochemical composition generates heat upon exposure to air.

13. The foot warming device of claim 12, wherein the airtight cover is removable to expose the air permeable container to air.

14. The foot warming device of claim 12, wherein the sock component comprises at least one pocket for retaining the warming component.

15. The foot warming device of claim 12, wherein the warming component is removable from the sock component.

16. The foot warming device of claim 12, wherein the thermochemical composition is comprises iron powder, water, a heat distributing material, a catalyst, and an insulator.

17. The foot warming device of claim 16, wherein the catalyst is NaCl.

18. The foot warming device of claim 17, wherein the insulator is vermiculite.

19. The foot warming device of claim 18, wherein the heat distributing material is activated carbon.

20. A foot warming device to improve blood flow to a foot comprising:
a sock component;
a plurality of warming components adapted for positioning within the sock component, each warming component comprising an air permeable container, an exothermic thermochemical composition retained within the air permeable container, an airtight base layer positional between the air permeable container and an inner surface of the sock component, and an airtight cover configured to encapsulate the air permeable container between the airtight cover and the airtight base layer; and
wherein the thermochemical composition generates heat upon exposure to air; and
wherein a first of the plurality of warming components is positioned adjacent to a heel of the sock component, and a second of the plurality of warming components is positioned adjacent to a sole of the sock component.
